(19) United States
(12) Patent Application Publication
(10) Pub. No.: US 2023/0157936 A1
(43) Pub. Date: May 25, 2023

(54) ANTI-AGING SKIN PEEL COMPOSITION AND METHOD OF APPLICATION

(71) Applicant: JAMRM, LLC, Dallas, TX (US)

(72) Inventors: Alisar Zahr, Dallas, TX (US); Thu Q. Nguyen, North Richland Hills, TX (US); John Koch, Fort Worth, TX (US); Tatiana Kelly, Westlake, TX (US)

(21) Appl. No.: 17/535,430

(22) Filed: Nov. 24, 2021

(51) Int. Cl. A61K 8/368; A61K 8/365; A61Q 19/08; A61K 8/362

(52) U.S. Cl. CPC A61K 8/368; A61K 8/362; A61K 8/365; A61Q 19/08

(57) ABSTRACT

A skincare composition and a method for removing layers of skin in a cosmetic procedure. The skincare composition comprises active ingredients mixed into a cosmetically acceptable carrier. The active ingredients include an effective amount of one or more keratolytic agents for inducing a desired degree of keratolysis in the skin, an effective amount of one or more free radical neutralizing agents for negating cellular damage from reactive chemical species, and an effective amount of one or more agents increasing synthesis of dermal components. The one or more free radical neutralizing agents and the one or more agents increasing synthesis of dermal components are acids in their un-neutralized, natural acid form, and a total amount of the active ingredients is less than 50 w/w% of the skincare composition, and a pH of the skincare composition is between 1.5-3.5.

### BACKGROUND

### Technical Field

Novel aspects of the present disclosure relate to the field of skin care and more particularly to a method and corresponding skincare composition providing initial controlled, superficial wounding of the skin along with delivery of beneficial free radical neutralizing agents and agents increasing synthesis of dermal components while the skin barrier is compromised.

### Background

Exfoliating and rejuvenating the skin has a very long history. Early records show that ancient Egyptians applied sour milk and other substances, such as animal oils and alabaster, to their skin to improve appearance and texture. In Roman times, people would use fruits such as grapes to exfoliate their skin. Other treatments included poultices containing substances such as Sulphur, mustard, and limestone, which were used to fade freckles and to even the skin tone. These ancient records show us that skin care has been important, particularly to women, through much of history.

In 1874, a dermatologist in Vienna called Ferdinand von Hebra, used peeling techniques to treat pigmentation conditions such as melasma, freckles and Addison’s disease. In 1882 German dermatologist Paul G Unna described his research for treating skin diseases based on his investigation into the biochemical processes of the skin. It was following his reports that other authors started publishing their work. In 1917, during World War I, Douglass Montgomery described use of peeling agents under bandages for healing and ‘beautifying’ the skin. The use of chemical peels started to gain momentum in the 1960s, and in the 70s and 80s alpha hydroxy acids (AHAs) were introduced for more superficial treatment. Skin peels are commonly based upon acid technology. An acid is a molecule (or ion) capable of donating a proton. Common examples of acids include hydrochloric acid, acetic acid, sulfuric acid, and citric acid. The strength of an acid is described in terms of pH, which is an abbreviation of the term “potential of Hydrogen”. The pH continuum extends from 1 to 14 with acids having a pH below 7 and bases having a pH above 7. A solution with a pH of 7 is considered neutral, i.e., neither acidic nor basic. Stronger acids have lower pH values. Conversely, stronger bases have a higher pH.

Chemical peels are a means of rejuvenating the skin by exfoliating, peeling, or sloughing off the upper layer(s) of dead keratinocyte skin cells that sit on the skin’s epidermal surface and by stimulating the production of new healthy epidermal cells as well as components of the extra cellular matrix in the dermis. Chemical peels are designed to introduce a controlled injury to the skin at a specific depth. As the healing process occurs there is a rejuvenation effect and an improvement in the skin’s texture and appearance. Chemical peels can target the skin at different depths and are often referred to as either a superficial chemical peel, a medium chemical peel, or a deep chemical peel.

A superficial peel utilizes light cleansing and defatting of the skin as preparation, lower acid concentrations and strengths, a higher pH range, less penetrating solvents in the vehicle delivery system and less repeated application passes and skin exposure time. A medium-depth peel utilizes moderate cleansing and defatting of the skin, moderate acid concentrations and strengths, a lower pH range, more penetrating solvents in the vehicle delivery system and a greater number of repeated application passes and length of skin exposure time. A deep chemical peel utilizes aggressive cleansing and defatting of the skin, high acid concentrations and strengths, a very low pH range, an increased amount of solvents in the vehicle delivery system, many repeated application passes, and extended skin exposure time.

Some undesirable side effects of chemical peels include redness, swelling, itching, scabbing, scarring, post-inflammatory hyperpigmentation, and infection. The severity of side effects increases with the strength and depth of the chemical peel. Recovery time from chemical peel procedures also increases with the strength and depth of the chemical peel. For example, superficial chemical peels can be fully healed in 1 to 28 days, but deep chemical peels can take 3 to 6 months to fully heal.

### SUMMARY OF THE INVENTION

Novel aspects of the present disclosure are directed to a skincare composition that causes an initial controlled, superficial wounding of the skin to provide a desired peeling and exfoliation effect. The skincare composition includes active ingredients mixed into a cosmetically acceptable carrier. The active ingredients include an effective amount of one or more keratolytic agents for inducing a desired degree of keratolysis in the skin, an effective amount of one or more free radical neutralizing agents for negating cellular damage from reactive chemical species, and an effective amount of one or more agents increasing synthesis of dermal components. The one or more free radical neutralizing agents and the one or more agents increasing synthesis of dermal components are acids in their un-neutralized, natural acid form, and a total amount of the active ingredients is not more than 50 w/w% of the skincare composition and a pH of the skincare composition is between 1.5 - 3.5.

Novel aspects of the present disclosure are also directed to a method of exfoliating the top layer(s) of the skin in a cosmetic peeling procedure with a skincare composition, the method including the steps of preparing the skin by cleansing and defatting the skin, applying the skincare composition to the skin; maintaining the skincare composition on the skin for an effective time for exfoliation to occur; and reapplying the skincare composition and maintaining the skincare composition on the skin for the effective time to further exfoliate the skin surface. The skincare composition includes active ingredients mixed into a cosmetically acceptable carrier. The active ingredients include an effective amount of one or more keratolytic agents for inducing a desired degree of keratolysis in the skin, an effective amount of one or more free radical neutralizing agents for negating cellular damage from reactive chemical species, and an effective amount of one or more agents increasing synthesis of dermal components. The one or more free radical neutralizing agents and the one or more agents increasing synthesis of dermal components are acids in their un-neutralized, natural acid form, and a total amount of the active ingredients is not more than 50 w/w% of the skincare composition and a pH of the skincare composition is between 1.5 - 3.5.

Other aspects, embodiments and features of the invention will become apparent from the following detailed description of the invention when considered in conjunction with the accompanying figures. In the figures, each identical, or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

### BRIEF DESCRIPTION OF THE FIGURES

The novel features believed characteristic of the invention are set forth in the appended claims. The invention itself, however, as well as a preferred method of use, further objectives and advantages thereof, will be best understood by reference to the following detailed description of illustrative embodiments when read in conjunction with the accompanying figures, wherein:

FIG. 1 is a flowchart of a process for exfoliating the skin’s surface utilizing a cosmetic procedure with a skincare composition in accordance with an illustrative embodiment.

### DETAILED DESCRIPTION

Aged skin is characterized by the presence of fine lines, wrinkles, rough texture, laxity, dull appearance, and uneven skin tone. Additionally, over time, human skin cells produce less energy and more free radicals, and the level and activity of matrix metalloproteinases (MMPs), which degrade extracellular matrix (ECM) proteins increases, resulting in a loss of elasticity and firmness. Examples of ECM proteins include collagen and elastin.

Application of chemical peels to human skin can cause exfoliation of the outer layer, which removes dead skin cells and wounds the underlying skin tissue. Conventional chemical peels, which are formulated primarily with alpha-hydroxy acids and with beta-hydroxy acids to a lesser effect, operate by attacking the bonds between the dead skin cells at the skin surface and the underlying, living skin tissue. These chemical peels cause a protein denaturing effect that disrupts the skin’s keratin barrier, causing its breakdown. The breakdown allows for skin cells to slough off while triggering the release of proinflammatory cytokines and chemokines to activate a healing signal for the regeneration of new ECM proteins. This activity in the skin results in the reduction of fine lines and wrinkles, reduced skin laxity, an evening of the skin tone, and a fuller more radiant appearance of the skin. Further, the initial controlled, superficial wounding of the skin also subsequently results in a rejuvenating effect of the skin during the wound healing process.

The degree of penetration and activity of chemical peels is dependent upon a number of factors, including: the specific preparation of the skin (degree of cleansing and defatting the skin), the type and strength of the acids used, the pH of the formulation, the vehicle delivery system, the number of repeated application passes, and the length of time which the skin is exposed to the chemical peel. A superficial chemical peel penetrates the intraepidermal and dermal-epidermal junction and is ideally suited for treating surface level fine lines, acne, and uneven skin tone. A medium-depth chemical peel penetrates full thickness epidermis and into the papillary dermis and is ideally suited for treating deeper wrinkles, scars, and uneven skin tone. A deep chemical peel penetrates the full thickness epidermis, papillary dermis and mid-reticular dermis and is ideally suited for removing deep wrinkles, scars, melasma, and precancerous growths.

Beneficial skincare ingredients that could have otherwise helped to protect the underlying skin and facilitate the desirable rejuvenating effect are not included in peels because the challenging acidic environment results in instability and reduced functionality of those ingredients. Accordingly, beneficial skincare ingredients are often contained in a separate posttreatment product applied at an indeterminate time after the chemical peel procedure. However, these beneficial skincare ingredients have difficulty penetrating the skin’s barrier in order to fully function and regenerate the skin.

None of the prior art, to applicant’s knowledge, discloses a superficial to medium depth peel utilizing alpha hydroxy acids and beta hydroxy acids combined with beneficial, unique acids not normally in skincare compositions in their un-neutralized and natural acid form as most skincare compositions used in a daily skincare regimen are not tolerable at the low pH of chemical peels. The un-neutralized, natural acid form is more effective than the partially neutralized form found in pH balanced skincare compositions which are customarily used in typical skincare products. These unique acids have reactive oxygen/nitrogen species neutralizing properties as well as anti-aging bio-stimulatory properties such as collagen and elastin synthesis. These unique acids include and are not limited to phenolic compounds including but not limited to hydroxycinnamic acids and their derivatives, as well as dicarboxylic acids and triterpenoids including pentacyclic triterpene acids and their derivatives.

Novel aspects of the skincare composition described in this disclosure provide for a chemical peel that has a stable formulation and inclusion of beneficial reactive oxygen/nitrogen species neutralizing ingredients and antiaging additives, in their un-neutralized and natural acid forms, which can be immediately delivered into the skin during the initial, controlled, superficial wounding process when the skin barrier is compromised and when the beneficial additives can best penetrate into the skin. The improved skincare formulation can provide an enhanced rejuvenating effect during exfoliation of the surface of the skin beyond what is normally experienced through a typical skin peel alone. The skincare composition described herein includes additives that can diminish the activity of matrix metalloproteinases (MMP) in the skin, promote collagen and elastin production in the skin, and increase reactive oxygen/nitrogen species neutralizing activity in the skin.

In a non-limiting embodiment, the skincare composition includes a plurality of active ingredients mixed into a cosmetically acceptable carrier. The cosmetically acceptable carrier can be a semi-occlusive suspension or solution. The active ingredients are selected to produce the exfoliating and peeling of skin surface layers and protection of underlying, living skin tissue. The pH of the skincare composition is between 1.5-3.5, or more specifically between 1.75-2.75. In a more particular embodiment, the pH of the skincare composition is between 2.0-3.0.

In one embodiment, the active ingredients include a keratolytic agent for inducing a desired degree of keratolysis in the skin, a neutralizing agent for negating cellular damage from reactive chemical species, and agents increasing synthesis of dermal components. The keratolytic agent can include one or more alpha-hydroxy acids, one or more beta-hydroxy acids, or a mixture of alpha-hydroxy acids and beta-hydroxy acids. The alpha and beta hydroxy acids work to denature the skin for an exfoliation effect. The neutralizing agent(s) can include at least one dicarboxylic acid, at least one phenolic acid, or a mixture of dicarboxylic acid(s) and phenolic acid(s). The neutralizing agent(s) are responsible for quenching both nitrogen and oxygen free radicals. The agents increasing synthesis of dermal components can include at least one triterpene acids. These agents increasing synthesis of dermal components can upregulate collagen and elastin and/or downregulate MMPs. Each of these categories of active ingredients are described in more detail in the paragraphs that follow.

### Keratolytic Agent

A keratolytic agent is a cosmetically acceptable ingredient or mixture of ingredients included in a skincare composition for inducing keratolysis in the skin. Keratolytic agents can include AHAs, BHAs, or mixtures of AHAs and BHAs.

### Alpha-Hydroxy Acids

AHAs are natural acids. Examples of common AHAs include citric acid (found in citrus fruits), glycolic acid (found in sugar cane), lactic acid (found in sour milk and tomato juice), malic acid (found in apples), and tartaric acid (found in grapes). AHAs are believed to contribute to the efficacy of chemical peels by degrading the inter-cellular protein complexes (e.g., hemidesmosomes, desmosomes) between cells, thereby loosening corneocytes for removal, and thus increasing cellular proliferation of keratinocytes. This allows for the generation of a thicker epidermis.

AHAs include a carboxylic acid substituted with a hydroxyl group (i.e., OH group) on the adjacent carbon atom. Most AHAs fall into the family or either monocarboxylic or dicarboxylic acids. Glycolic acid and lactic acid are reproduced below as examples.

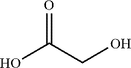

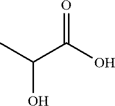

### Beta-Hydroxy Acids

Beta hydroxy acids (BHAs) are organic compounds containing a carboxylic acid functional group and a hydroxyl functional group separated by two carbon atoms. Examples of BHAs include, tropic acid, tethocanic acid, beta-hydroxypropionic acid, and salicylic acid.

The chemical formula for salicylic acid is shown below. The main purpose is for its anti-microbial properties as well as keratolytic properties. It is naturally found in willow bark.

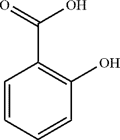

A non-limiting list of keratolytic agents that can be included in the novel skincare composition are provided in Table 1 below.

TABLE 1

Keratolytic Agents

| | |
|---|---|
| Acid Name | Acid Class |
| Glycolic Acid | AHA |
| Lactic Acid | AHA |
| Mandelic Acid | AHA |
| Malic Acid | AHA |
| Citric Acid | AHA/BHA |
| Salicylic Acid | BHA |
| Beta Hydroxybutanoic Acid | BHA |
| Tropic Acid | BHA |
| Trethocanic Acid | BHA |
| Carpryloyl Salicylic Acid | BHA |

### Agents Increasing Synthesis of Dermal Components

Agents increasing synthesis of dermal components are active ingredients that can enhance the rejuvenating effect of the skincare composition described in this disclosure. At least some of the agents increasing synthesis of dermal components enhance the rejuvenating effect by promoting collagen and elastin production in the skin, i.e., ECM proteins. Other agents increasing synthesis of dermal components can inhibit the activity of MMPs in the skin. MMPs are enzymes responsible for degrading components of the ECM. Thus, inhibition of these MMPs can help stimulate regeneration of skin by reducing the degradation of ECM proteins.

### Triterpene Acids

Triterpene acids, also referred to herein as triterpenoids, are biological active photo-chemicals and can be found in nature. They have unique properties can be found in many medicinal plants, such as Glycyrrhiza species, Gymnema species, Centella asiatica, Camellia sinensis, Crataegus species and Olea europaea, which are commonly used in traditional medicine for the treatment of diabetes and diabetic complications. A large number of bioactive pentacyclic triterpenoids, such as oleanolic acid, glycyrrhizin, glycyrrhetinic acid, ursolic acid, betulin, betulinic acid and lupeol have shown multiple biological activities including, upregulation of collagen, downregulation of matrix metalloproteinases (MMPs), inhibition of lipid peroxidation, improvement in cellular antioxidant defenses, and anti-inflammatory properties.

An example of a triterpene acid is ursolic acid, which is shown below.

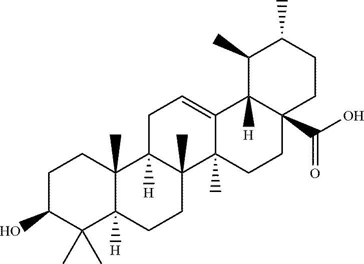

A non-limiting list of agents increasing synthesis of dermal components that can be included in the novel skincare composition are provided in Table 2 below.

TABLE 2

Agents increasing synthesis of dermal components

| | | |
|---|---|---|
| Acid Name | Acid Type | Mechanism of Action |
| Ursolic Acid | Pentacyclic Triterpene Acid | Down Regulates MMP 9; Inhibits Lipid Peroxidation |
| Asiatic Acid | Pentacyclic Triterpene Acid | Promotes Collagen I Production |
| Betulinic Acid | Pentacyclic Triterpene Acid | Inhibits MMP 2 and MMP 9; Overexpression of TIMP-2 |
| Oleanolic Acid | Pentacyclic Triterpene Acid | Inhibits Lipid Peroxidation; Promotes Collagen Production; Reduces MMP 3 |
| Madecassic Acid | Triterpene Acid | Promotes Collagen I Production |

### Free Radical Neutralizing Agents

Free radical neutralizing agents are active ingredients selected for negating cellular damage from reactive chemical species. The most common reactive chemical species are reactive oxygen species (ROS) and reactive nitrogen species (RNS). ROS are highly reactive chemicals formed from O2, examples of which include peroxides, superoxides, hydroxyl radicals, singlet oxygen, and alpha-oxygen. RNS are also highly reactive chemicals but formed from a reaction of nitric oxide with superoxide to form peroxynitrite. Damage initiated by ROS/RNS is thought to be a major contributor to cellular aging. Free radical neutralizing agents are capable of detoxifying one or more ROS/RNS subspecies according to known mechanisms. Some phenolic acids and some dicarboxylic acids can serve as free radical neutralizing agents.

### Phenolic Acids

Phenolic acids, also known as phenolcarboxylic acids, are aromatic acid compounds. Examples of aromatic acid compounds include substances containing a phenolic ring and an organic carboxylic acid function (C6-C1 skeleton). Two naturally occurring types of phenolic acids are hydroxybenzoic acids and hydroxycinnamic acids, which are derived from non-phenolic molecules of benzoic acid and cinnamic acid, respectively. Examples of phenolic acids are caffeic acid and gallic acid, which are shown below.

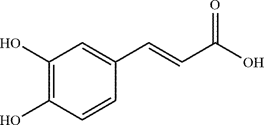 [SMILES: O=C(O)/C=C/c1ccc(O)c(O)c1]

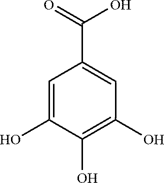 [SMILES: O=C(O)c1cc(O)c(O)c(O)c1]

### Dicarboxylic Acids

Dicarboxylic acids are organic compounds containing two carboxyl functional groups (—COOH). The general molecular formula for dicarboxylic acids is HOOC—R—COOH, where R can be aliphatic or aromatic. Dicarboxylic acids show similar chemical behavior and reactivity to monocarboxylic acids. Dicarboxylic acids are often used in the preparation of copolymers such as polyamides and polyesters. The most widely used dicarboxylic acids is adipic acid, which is used in the natural food industry for flavor enhancing. Other examples of dicarboxylic acids include azelaic acid, aspartic acid and glutamic acid, both of which are amino acids in the human body. An example of a dicarboxylic acids that can serve as a neutralizing agent is azelaic acid, shown below.

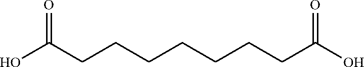 [SMILES: O=C(O)CCCCCCCC(=O)O]

A non-limiting list of free radical neutralizing agents that can be included in the novel skincare composition are provided in Table 3 below.

TABLE 3

Free radical neutralizing agents

| | | |
|---|---|---|
| Acid Name | Acid Type | Mechanism of Action |
| Tartaric Acid | Dicarboxylic Acid | Scavenges Reactive Nitrogen Species, Nitric Oxide; |
| Azelaic Acid | Dicarboxylic Acid | Scavenges Reactive Oxygen Species |
| Nordihydroguaiaretic Acid | Phenolic Acid | Scavenges Reactive Oxygen Species, Singlet Oxygen, Super Oxide Anion, Hydroxyl Radical, Peroxynitrite, Hypochlorous Acid |
| Gallic Acid | Phenolic Acid | Scavenges Reactive Nitrogen Species, Hydrogen Peroxide, Hydroxyl Radicals |
| Caffeic Acid | Phenolic Acid | Scavenges Super Oxide Anion |

### Example(s)

In a non-limiting example, the skincare composition for exfoliating and peeling the skin’s surface in a cosmetic procedure includes active ingredients mixed into a cosmetically acceptable carrier. The active ingredients include an effective amount of one or more keratolytic agents for inducing a desired degree of keratolysis in the skin, an effective amount of one or more free radical neutralizing agents for negating cellular damage from reactive chemical species, and an effective amount of one or more agents increasing synthesis of dermal components. As used herein, the phrase “effective amount” means a sufficient amount of a compound that can significantly induce a positive modification the condition being treated, but low enough to avoid unwanted side effects, within the scope of sound judgment of a skilled artisan. The effective amount of a compound may vary with the particular condition being treated, the age and condition of the biological subject being treated, the severity of the condition, the duration of the treatment, and other factors within the knowledge and expertise of the skilled artisan. In this non-limiting example, a total amount of the active ingredients is less than 30 (w/w)t% of the skincare composition, and a pH of the skincare composition is between 1.5-3.5.

In this non-limiting example, the keratolytic agents can be selected from any one or more acids selected from the keratolytic agents listed in Table 1. When the keratolytic agent is one or more AHAs, the effective amount of AHAs is between 4-70 w/w% of the skincare composition, or more particularly between 7-60 w/w%. In a particular embodiment of this non-limiting example, the effective amount of the keratolytic agent formed from one or more AHAs is about 20.1 w/w% of the skincare composition. In one or more embodiments of this non-limiting example, a concentration of each of the one or more AHAs is less than 12 w/w% of the cosmetic formulation, or more particularly less than 11 w/w% of the cosmetic formulation. In a more particular embodiment, the concentration of each of the AHAs is not greater than 10 w/w%.

When the keratolytic agent includes one or more BHAs and/or their derivatives, selected from the list of keratolytic agents in Table 1, the effective amount of the one or more BHAs is between 0.2-0.3 w/w% of the skincare composition, or more particularly between 0.225-0.275 w/w%. In a particular embodiment of this non-limiting example, the effective amount of the one or more BHAs is about 0.25 w/w%. In one or more embodiments of this non-limiting example, a concentration of each of the one or more BHAs is less than 1 w/w% of the cosmetic formulation, or more particularly less than 0.5 w/w% of the cosmetic formulation. In a more particular embodiment, the concentration of each of the BHAs is not greater than 0.25 w/w%.

In this non-limiting example, the agents increasing synthesis of dermal components are triterpene acids and their derivatives, which can be selected from Table 2. The effective amount of the one or more agents increasing synthesis of dermal components is between 0.01-3.0 w/w% of the skincare composition, or more particularly between 0.01-1.5 w/w%. In a particular embodiment of this non-limiting example, the effective amount of the one or more triterpene acids is about 0.01 w/w%.

In this non-limiting example, free radical neutralizing agents include the phenolic acids and their derivatives, and dicarboxylic acids and their derivatives, which can be selected from the acids presented in Table 3. When the free radical neutralizing agents include phenolic acids and their derivatives, the effective amount of the one or more phenolic acids is between 0.005-3.5 w/w% of the cosmetic formulation, or more particularly between 1-2.5 w/w%. In a particular embodiment of this non-limiting example, the effective amount of the one or more phenolic acids is about 1.75 w/w% of the cosmetic formulation.

In this non-limiting example, when the free radical neutralizing agents include dicarboxylic acids and their derivatives, the effective amount of the one or more dicarboxylic acids is between 0.15-15 w/w% of the cosmetic formulation, or 3-7 w/w% of the cosmetic formulation, or more particularly between 4-6 w/w%. In a particular embodiment of this non-limiting example, the effective amount of the one or more dicarboxylic acids is about 5.1 w/w% of the cosmetic formulation. In one or more embodiments of this non-limiting example, a concentration of each of the one or more dicarboxylic acids is less than 3 w/w% of the cosmetic formulation, or more particularly less than 2.75 w/w% of the cosmetic formulation. In a more particular embodiment, the concentration of each of the dicarboxylic acids is not greater than 2.5 w/w%.

In another non-limiting example, the skincare composition includes active ingredients mixed into a cosmetically acceptable carrier. The active ingredients include an effective amount of one or more AHAs, an effective amount of one or more BHAs, an effective amount of one or more pentacyclic triterpene acids, an effective amount of one or more phenolic acids, and an effective amount of one or more dicarboxylic acids. In this other non-limiting example, a total amount of the active ingredients is less than 30 w/w% of the skincare composition, and a pH of the skincare composition is between 1.5-3.5.

In yet another non-limiting example, the skincare composition includes active ingredients mixed into a cosmetically acceptable carrier. The active ingredients include an effective amount of one or more AHAs, an effective amount of one or more BHAs, and an effective amount of one or more dicarboxylic acids. In this other non-limiting example, a total amount of the active ingredients is less than 30 w/w% of the skincare composition, and a pH of the skincare composition is between 1.5-4.

In this other non-limiting example, the AHAs can include one or more of the following: glycolic acid, lactic acid, mandelic acid, and citric acid. Further, the effective amount of AHAs is between 15-25 w/w% of the skincare composition, or more particularly between 17.5-22.5 w/w%. In a particular embodiment of this non-limiting example, the effective amount of the AHAs is about 20.1 w/w% of the skincare composition. In one or more embodiments of this non-limiting example, a concentration of each of the one or more AHAs is less than 12 w/w% of the cosmetic formulation, or more particularly less than 11 w/w% of the cosmetic formulation. In a more particular embodiment, the concentration of each of the AHAs is not greater than 10 w/w%.

In this other non-limiting example, the dicarboxylic acids can include one or more of malic acid, azelaic acid, and tartaric acid. Further, the effective amount of the one or more dicarboxylic acids is between 3-7 w/w% of the cosmetic formulation, or more particularly less than 4-6 w/w%. In a particular embodiment of this non-limiting example, the effective amount of the one or more dicarboxylic acids is about 5.1 w/w% of the cosmetic formulation. In one or more embodiments of this non-limiting example, a concentration of each of the one or more dicarboxylic acids is less than 3 w/w% of the cosmetic formulation, or more particularly less than 2.75 w/w% of the cosmetic formulation. In a more particular embodiment, the concentration of each of the dicarboxylic acids is not greater than 2.5 w/w%.

In this other non-limiting example, the BHAs can include one or more of salicylic acid and carpryloyl salicylic acid. Further, the effective amount of the one or more BHAs is between 0.2-0.3 w/w% of the skincare composition, or more particularly between 0.225-0.275 w/w%. In a particular embodiment of this non-limiting example, the effective amount of the one or more BHAs is about 0.25 w/w%. In one or more embodiments of this non-limiting example, a concentration of each of the one or more BHAs is less than 1 w/w% of the cosmetic formulation, or more particularly less than 0.5 w/w% of the cosmetic formulation. In a more particular embodiment, the concentration of each of the BHAs is not greater than 0.25 w/w%.

In one or more of the previous examples, or another example, the one or more dicarboxylic acids is azelaic acid, which is selected for free radical scavenging of reactive oxygen species.

In one or more of the previous examples, or another example, the one or more dicarboxylic acids is tartaric acid, the one or more phenolic acids is gallic acid, and the tartaric acid and the gallic acid are selected for free radical scavenging of reactive nitrogen species.

In one or more of the previous examples, or another example, the one or more phenolic acids is a mixture of gallic acid and caffeic acid and the one or more pentacyclic triterpene acids is oleanolic acid, and the gallic acid, the caffeic acid, and the oleanolic acid are selected for increasing collagen production in the skin.

In one or more of the previous examples, or another example, the one or more pentacyclic triterpene acids is oleanolic acid, which is selected for inhibiting lipid peroxidation and inhibiting matrix metalloproteinase (MMP) activity in the skin.

In one or more of the previous examples, the skincare composition lacks resorcinol. Resorcinol can provide exfoliating effects, but prolonged exposure at high concentrations can cause more severe side-effects including systemic toxicity and hyperthyroidism. Thus, the skincare composition described herein avoids one common health risk associated with a common chemical peel ingredient.

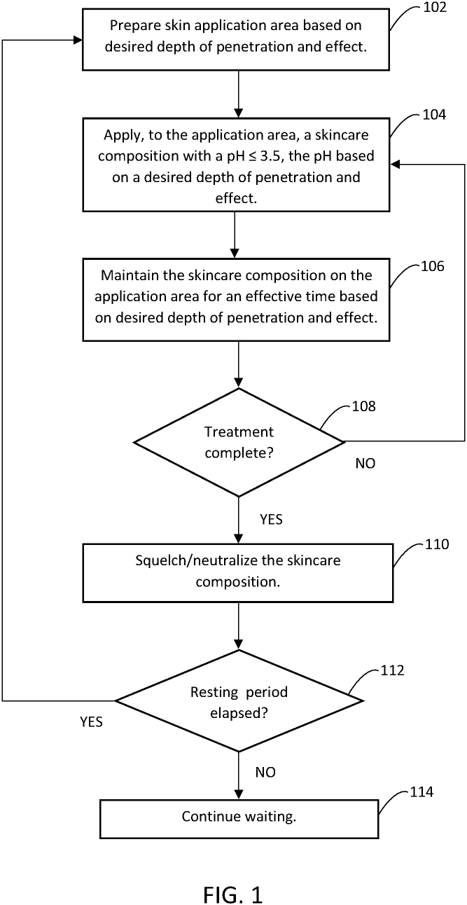

FIG. 1 is a flowchart of a process for removing layers of skin in a cosmetic procedure according to an illustrative embodiment. The steps of flowchart 100 can be carried out on a patient by a skincare professional, such as a doctor, nurse, or aesthetician. An example of the cosmetic procedure is a chemical skin peel. Further, the method can be used on the patient during a first encounter of treatment, i.e., a visit, with further encounters requiring more treatment passes with a maximum of four total passes.

Flowchart 100 begins at Step 102 by preparing the application area based on a desired depth of penetration and effect. In a non-limiting embodiment, preparation of the application area is achieved by cleansing and defatting the application area. More aggressive cleansing provides a greater depth of penetration of the skincare composition and a stronger effect, i.e., degree of exfoliation. The most common application area is the skin on the face. However, the application area can also be the neck, or other areas of skin that frequently exhibits signs of aging, such as the hands.

In Step 104, the skincare composition is applied to the application area. A pH of the skincare composition is less than or equal to 3.5, but the actual pH is based on the desired depth of penetration and effect. Lower pH provides a greater depth of penetration of the skincare composition and a stronger effect. In some embodiments, the range of acceptable pH for the skincare composition can be between 1.5 and 3.5, modified by increasing the amount of one or more of the active ingredients to lower the pH, e.g., AHAs, BHAs, or other acid ingredients. As previously discussed, the skincare composition includes active ingredients mixed into a cosmetically acceptable carrier. The active ingredients include an affective amount of one or more AHAs, an effective amount of one or more BHAs, an effective amount of one or more pentacyclic triterpene acids, an effective amount of one or more phenolic acids, and an effective amount of one or more dicarboxylic acids. In one embodiment, the total amount of the active ingredients is less than 50 w/w% of the skincare composition. More particularly, the total amount of the active ingredients is less than 40 w/w% of the skincare composition, and in a particular embodiment the total amount of the active ingredients is less than 30 w/w% of the skincare composition.

In Step 106, the skincare composition is maintained on the application area for an effective time. The effective time is based on a desired depth of penetration and effect. Longer times provide a greater depth of penetration of the skincare composition and a stronger effect. In a non-limiting embodiment, the effective time to exfoliate the skin is between 1 - 5 minutes, and more particularly between 1.5 - 3 minutes. In a particular embodiment, the effective time to exfoliate the skin is about 2 minutes.

In Step 108 a determination is made as to whether the treatment is complete. If the treatment is not complete, then flowchart 100 returns to Step 104. In some embodiments, the treatment is complete once the desired number of treatment passes has been performed. Greater number of treatment passes provides a greater depth of penetration of the skincare composition and a stronger effect. If the treatment is complete, then flowchart 100 proceeds from Step 108 to Step 110 where the skincare composition is squelched/neutralized. A conventional neutralizer can be applied which counteracts the acidity of the skincare composition to cease wounding of the skin. Step 110 can also include other additional steps known to those skilled in the art, such as a cleansing routine with cool water.

The efficacy of the skincare procedure described in flowchart 100 increases with increasing number of applications and by targeting incrementally deeper layers of skin tissue. Thus, in a non-limiting embodiment, the cosmetic procedure described in flowchart 100 is repeated over at least three visits. For example, in the first visit, the cosmetic procedure exfoliates the skin. In the second visit, the reapplying and maintaining steps can be repeated so that cosmetic procedure exfoliates further layers of the skin. In the third visit, the reapplying and maintain steps can be repeated so that the cosmetic procedure further exfoliates the skin. For optimum results and to allow the skin sufficient time to heal, each visit should be spaced apart for a resting period of about 2-6 weeks, or more particularly between 2-5 weeks. Accordingly, flowchart 100 includes determining, in Step 112, whether the resting period has elapsed before the next treatment cycle begins again. If the resting period has elapsed, then flowchart 100 proceeds to Step 102 where the process begins again. However, if the resting period has not yet elapsed, then flowchart 100 proceeds to Step 114 until the resting period has elapsed.

Although embodiments of the invention have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, or composition for an element or process for making or using an element or combination of elements, the characteristics, structure, size, shape, arrangement, or composition can also be incorporated into any other element or combination of elements, or process for making or using an element or combination of elements described herein to provide additional embodiments.

Additionally, where an embodiment is described herein as comprising some element or group of elements, additional embodiments can consist essentially of or consist of the element or group of elements. Also, although the open-ended term “comprises” is generally used herein, additional embodiments can be formed by substituting the terms “consisting essentially of” or “consisting of.”

While this invention has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A skincare composition for use in exfoliation of skin, the skincare composition comprising:
active ingredients mixed into a cosmetically acceptable carrier, wherein:
the active ingredients comprise (a) an effective amount of a keratolytic agent for inducing a desired degree of keratolysis in the skin, (b) an effective amount of one or more free radical neutralizing agents for negating cellular damage from reactive chemical species, and (c) an effective amount of one or more agents increasing the synthesis of dermal components;
the one or more free radical neutralizing agents and the one or more agents increasing synthesis of dermal components are acids in an un-neutralized, natural acid form;
a pH of the skincare composition is between 1.5 - 3.5; and
a total amount of the active ingredients is less than 50 w/w% of the skincare composition.

2. The skincare composition of claim 1, wherein the keratolytic agent is a mixture of an effective amount of one or more alpha-hydroxy acids (AHAs) and an effective amount of one or more beta-hydroxy acids (BHAs).

3. The skincare composition of claim 2, wherein:
the effective amount of the AHA is 5 - 60 w/w% of the active ingredients; and
the effective amount of the BHA is less than 10 w/w% of the active ingredients.

4. The skincare composition of claim 3, wherein:
a concentration of each of the one or more AHAs is less than 20 w/w% of the active ingredients; and
a concentration of each of the one or more BHAs is less than 5 w/w% of the active ingredients.

5. The skincare composition of claim 2, wherein:
the one or more AHAs comprises at least one of glycolic acid, lactic acid, mandelic acid, and citric acid;
the one or more BHAs comprises at least one of salicylic acid, tropic acid, and trethocanic acid.

6. The skincare composition of claim 1, wherein:
the one or more free radical neutralizing agents comprise at least one dicarboxylic acid and at least one phenolic acid; and
the one or more agents increasing synthesis of dermal components comprise at least one triterpene acid.

7. The skincare composition of claim 6, wherein:
the at least one dicarboxylic acid comprises at least one of malic acid, azelaic acid, and tartaric acid;
the at least one phenolic acid comprises at least one of gallic acid, caffeic acid, and nordihydroguaiaretic acid; and
the at least one triterpene acid comprises at least one of Asiatic acid, betulinic acid, oleanic acid, ursolic acid, and madecassic acid.

8. The skincare composition of claim 6, wherein:
the effective amount of the one or more free radical neutralizing agents comprises an effective amount of the at least one dicarboxylic acid and an effective amount of the at least one phenolic acid;
the effective amount of the at least one dicarboxylic acid is between 0.15 - 15 w/w% of the active ingredients;
the effective amount of the at least one phenolic acid is between 0.005 - 3.5 w/w% of the active ingredients; and
the effective amount of the at least one triterpene acid is between 0.010-3.0 w/w% of the active ingredients.

9. The skincare composition of claim 8, wherein:
a concentration of each of the one or more dicarboxylic acids is less than 5 w/w%;
a concentration of each of the one or more phenolic acids is less than 1 w/w%; and
a concentration of each of the one or more triterpene acids is less than 1 w/w%.

10. The skincare composition of claim 1, wherein the active ingredients lack resorcinol.

11. A method for exfoliating the skin in a cosmetic procedure with a skincare composition, the method comprising:
applying the skincare composition to the skin that has been cleansed and defatted;
maintaining the skincare composition on the skin for an effective time to exfoliate the surface of the skin; and
reapplying the skincare composition and maintaining the skincare composition on the skin for the effective time to remove another layer of skin,
wherein the skincare composition includes active ingredients mixed into a cosmetically acceptable carrier, wherein:
the active ingredients comprise (a) an effective amount of a keratolytic agent for inducing a desired degree of keratolysis in the skin, (b) an effective amount of one or more free radical neutralizing agents for negating cellular damage from reactive chemical species, and (c) an effective amount of one or more agents increasing synthesis of dermal components;
the one or more free radical neutralizing agents and the one or more agents increasing synthesis of dermal components are acids in an un-neutralized, natural acid form;
a pH of the skincare composition is between 1.5 - 3.5; and
a total amount of the active ingredients is less than 50 w/w% of the skincare composition.

12. The method of claim 11, wherein the reapplying step and the maintaining step are repeated to remove three layers of skin in a second visit.

13. The method of claim 12, wherein the reapplying step and the maintaining step are repeated to remove four layers of skin in a third visit.

14. The method of claim 11, wherein maintaining the skincare composition on the skin for the effective time further comprises:
removing the skincare composition after the effective time with a cleanser.

15. The method of claim 11, wherein the keratolytic agent is a mixture of an effective amount of one or more alpha-hydroxy acids (AHAs) and an effective amount of one or more beta-hydroxy acids (BHAs).

16. The method of claim 15, wherein:
the effective amount of the AHA is 5 - 60 w/w% of the active ingredients; and
the effective amount of the BHA is less than 10 w/w% of the active ingredients.

17. The method of claim 16, wherein:
a concentration of each of the one or more AHAs is less than 20 w/w% of the active ingredients; and
a concentration of each of the one or more BHAs is less than 5 w/w% of the active ingredients.

18. The method of claim 15, wherein:
the one or more AHAs comprises at least one of glycolic acid, lactic acid, mandelic acid, and citric acid;
the one or more BHAs comprises at least one of salicylic acid, tropic acid, and trethocanic acid.

19. The method of claim 11, wherein:
the one or more free radical neutralizing agents comprise at least one dicarboxylic acid and at least one phenolic acid; and
the one or more agents increasing synthesis of dermal components comprise at least one triterpene acid.

20. The method of claim 19, wherein:
the at least one dicarboxylic acid comprises at least one of malic acid, azelaic acid, and tartaric acid;
the at least one phenolic acid comprises at least one of gallic acid, caffeic acid, and nordihydroguaiaretic acid; and
the at least one triterpene acid comprises at least one of Asiatic acid, betulinic acid, oleanic acid, ursolic acid, and madecassic acid.
